(19) United States
(12) Patent Application Publication
(10) Pub. No.: US 2021/0393139 A1
(43) Pub. Date: Dec. 23, 2021

(54) System And Device For The Contactless Measure Of The Body Temperature Of A Person

(71) Applicants: Alessandro Manneschi, Arezzo (IT); Luca Manneschi, Arezzo (IT)

(72) Inventors: Alessandro Manneschi, Arezzo (IT); Luca Manneschi, Arezzo (IT)

(21) Appl. No.: 17/352,853

(22) Filed: Jun. 21, 2021

(30) Foreign Application Priority Data
Jun. 22, 2020 (FR) 2006484

(51) Int. Cl. A61B 5/01; A61B 5/00; A61B 5/1171

(52) U.S. Cl. CPC A61B 5/01; A61B 5/0008; A61B 5/0075; A61B 2562/0271; A61B 5/1176; A61B 2560/0223; A61B 5/0077

(57) ABSTRACT

The present invention concerns a measurement system (10) of an individual's body temperature comprising: an archway (11) delimiting a passageway (9) for an individual;

a visible spectrum camera (6) fixed onto the archway (11) so that a field of view (31) of the visible spectrum camera (6) covers at least a portion of the passageway (9);

an infrared camera (12) fixed onto the archway (11) so that a field of view (32) of the infrared camera (12) covers all or part of the portion of the passageway (9); and

a processing unit (15) configured to identify the pixels corresponding to the individual's face in the visible image, to determine the corresponding pixels in the electronic image and to determine a maximum temperature value associated with the pixels corresponding to the face in order to deduce therefrom a body temperature of the individual.

### FIELD OF THE INVENTION

The invention generally concerns measuring the body temperature of an individual, especially at the entrance to a limited access area, such as a public or private building.

### STATE OF THE ART

The current health situation has demonstrated the necessity of being able to quickly check the body temperature of individuals wishing to have access to a particular area, such as the entrance of a public or private building. In particular, it is desirable to be able to identify people whose body temperature is greater than 37.5° C. in order to limit the risk of spreading viruses (such as the coronavirus COVID-19).

In the medical field, portable thermometers are known for checking the temperature with contact, which has the advantage of being economical and precise. However, these thermometers require physical contact with the skin of the individual to be checked, which involves a relatively long measurement time and the need to replace a thermometer insulation capsule each time in order to ensure the hygiene of the test.

There are also gun-type portable infrared control systems. These systems allow an operator to measure the temperature of an individual without contact, by being placed near the individual to be checked. The temperature is generally measured at the forehead or wrists of the individual, very quickly. However, since the system is in a gun form, it must be handled by a dedicated operator. Moreover, the precision of the infrared sensor varies over time due to environmental thermal variations and the drift of the characteristics of the infrared sensors. To improve the precision of the system, it would be necessarily to calibrate it periodically, which is cumbersome for a portable system of this type. Moreover, the distribution of the temperature over the face varies depending on its exposure to the external environment (for example, wind or sun) and according to the individual. However, an operator cannot determine beforehand the area of the face having the maximum temperature, so that the temperature measured does not necessarily correspond to the actual body temperature of the individual.

There are also fixed installation infrared checkpoint systems, comprising an infrared camera mounted on a support, whose optical axis is parallel to the ground and at the average height of a face. However, these systems have the same gaps as the portable systems while, since they are fixed, their operation can be more complex to improve the measurement precision. In particular, the behavior of the infrared sensors of these fixed systems is stabilized by a thermal control of the temperature measured by the infrared sensor and by the periodic and sometimes continuous use of an external black body used as a reference whose emissivity is unitary and whose temperature is known. However, it results that the use of an external black body often poses installation problems that can make the installation of a checkpoint system complex. Moreover, the black body may experience interference (individuals passing by, for example). Moreover, an operator must manually manage the moment when the individual whose body temperature is to be determined is found at an appropriate distance to perform the measurement and synchronize reading the temperature with the instant the individual passes. Finally, when measurements are performed at the entrance to an area of limited access, it appears difficult to isolate the individual whose temperature is being measured and ignore individuals passing alongside, since these individuals also form a heat source and are therefore likely to disrupt the measurements.

### DISCLOSURE OF THE INVENTION

One objective of the invention is to remedy the above-mentioned disadvantages.

In particular, one objective of the invention is to propose a contactless measurement system for an individual's body temperature that is reliable, stable over time and provides a precise value of the individual's body temperature, regardless of the measurement environment.

Another objective of the invention is to propose a measurement system for an individual's body temperature that can be easily transported by an operator and set up rapidly in a given access and that does not require handling by the operator when measuring the temperature.

Another objective of the invention is to propose a measurement system that makes it possible to limit environmental disruptions, despite the presence of any individuals other than the one whose body temperature is to be determined.

For this purpose, according to a first aspect of the invention, a measurement of an individual's body temperature is proposed comprising:
an archway comprising two side panels connected by a cross member and together delimiting a passageway for an individual;
a visible spectrum camera comprising a visible spectrum detection chip comprising a visible pixel matrix and being configured to generate a visible image comprising a plurality of visible image pixels, the visible spectrum camera being fixed onto the archway so that a field of view of the visible spectrum camera covers at least a portion of the passageway;
an infrared camera comprising an infrared detection chip comprising an infrared pixel matrix and being configured to convert the infrared radiation received by each infrared pixel into a corresponding temperature value and to generate an electronic image comprising a plurality of infrared image pixels, each infrared image pixel being representative of the temperature value received by a corresponding infrared pixel, the infrared camera being fixed onto the archway so that a field of view of the infrared camera covers all or part of the portion of the passageway; and
a processing unit configured to identify the visible image pixels corresponding to at least a part of the individual's face, to match the visible image and the electronic image in order to identify the infrared image pixels corresponding to the visible image pixels identified, to determine a maximum temperature value associated with the identified infrared pixels and to deduce therefrom a body temperature of the individual.

Some preferred but non-limiting characteristics of the measurement system according to the first aspect are as follows, taken individually or in combination:
the measurement system also comprises a calibration module comprising:
a first reference target and a second reference target positioned in the field of view of the infrared camera so that the electronic image comprises infrared image pixels representative of the temperature value of the first reference target and the second reference target, and
a first thermal sensor configured to measure an instantaneous temperature of the first reference target and a second thermal sensor configured to measure an instantaneous temperature of the second reference target,
the processing unit also being configured to determine a gain coefficient and an offset coefficient from the respective instantaneous temperatures of the first reference target and the second reference target and the temperature values of the first reference target and the second reference target in the electronic image and to apply the gain coefficient and the offset coefficient determined to the temperature value associated with each infrared image pixel so as to obtain a corrected electronic image, the processing unit being configured to determine the maximum temperature value from the corrected electronic image;
   the processing unit is also configured to apply a predetermined compensation coefficient to the temperature value of each infrared image pixel;
   the measurement system also comprises a presence sensor configured to determine a presence of an individual in the passageway;
   the first reference target and the second reference target are fixed onto one of the two side panels and the cross member;
   the first reference target has a first reference temperature and the second reference target has a second reference temperature different from the first reference temperature;
   the first reference temperature and the second reference temperature are comprised between 35° C. and 40° C.;
   the measurement system also comprises a first heating element and a second heating element configured to maintain the first reference target and the second reference target at the first reference temperature and at the second reference temperature; respectively;
   the measurement system also comprises a signalling unit configured to generate an alert when the maximum temperature value is greater than a predetermined threshold;
   the infrared camera and the visible spectrum camera are mounted on an arm fixed onto the cross member and extending from an exit of the archway;
   the processing unit is configured to identify the visible image pixels corresponding to the individual's eyes, preferably an inner corner of at least one eye of the individual; and/or
   the measurement system also comprises a light, preferably flashing, fixed near the infrared camera so as to draw the gaze of an individual passing through the passageway.

According to a second aspect, the invention proposes a method for measuring an individual's body temperature by means of a measurement system according to the first aspect, comprising the following steps:

S1: producing an electronic image of a portion of the passageway in which an individual is located, the electronic image comprising a plurality of infrared image pixels representative of a temperature value received by a corresponding infrared pixel of a matrix of infrared pixels of an infrared camera;

S2: producing a visible image of all or part of the portion of the passageway, the visible image comprising a plurality of visible image pixels;

S3: identifying the visible image pixels corresponding to at least a part of an individual's face, preferably at least one inner corner of the eyes;

S4: matching the visible image and the electronic image so as to identify the infrared image pixels corresponding to the visible image pixels identified in step S3;

S9: determining a maximum temperature value associated with the identified infrared image pixels and deducing therefrom a body temperature of the individual.

Some preferred but non-limiting characteristics of the measurement method according to the second aspect are as follows, taken individually or in combination:
steps S1 and S2 are simultaneous;
the method also comprises the following steps, prior to step S9:

S5: determining an instantaneous temperature of the first reference target and the second reference target;

S6: determining in the electronic image the temperature value of the image pixels corresponding to the first reference target and to the second reference target;

S7: deducing therefrom a gain coefficient and an offset coefficient for the infrared camera; and

S8: applying the gain coefficient and the offset coefficient to the temperature value associated with each image pixel so as to obtain a corrected electronic image;
the method also comprises the application of a predetermined compensation coefficient to the temperature value of each image pixel or to the maximum temperature value determined in step S9;
the method also comprises, prior to step S1, a step S0 of determination of a presence of an individual in the archway, the steps S1 to S9 being implemented only when an individual is present in the passageway;
the method also comprises an alert generation step when the maximum temperature value is greater than a predetermined threshold; and/or
during step S3, the visible image pixels corresponding to the individual's eyes are identified, preferably visible image pixels corresponding to at least one inner corner of the eyes of the individual.

According to a third aspect, the invention proposes a system for measuring an individual's body temperature comprising:
an archway comprising two side panels connected by a cross member and together delimiting a passageway for an individual;
an infrared camera comprising an infrared detection chip comprising an infrared pixel matrix and being configured to convert the infrared energy received by each pixel of the matrix into a corresponding temperature value and to generate an electronic image comprising a plurality of infrared image pixels, each infrared pixel being representative of the temperature value received by a corresponding pixel of the matrix, the infrared camera being fixed to the archway so that a field of view of the infrared camera covers at least a portion of the passageway;
a first reference target positioned in the field of view of the infrared camera so that the electronic image comprises image pixels representative of the temperature value of the first reference target;
a second reference target positioned in the field of view of the infrared camera so that the electronic image comprises image pixels representative of the temperature value of the second reference target;
a first thermal sensor configured to measure an instantaneous temperature of the first reference target;
a second thermal sensor configured to measure an instantaneous temperature of the second reference target; and
a processing unit configured to determine a gain coefficient and an offset coefficient from the respective instantaneous temperatures of the first reference target and the second reference target and the temperature values of the first reference target and the second reference target in the electronic image and to apply the gain coefficient and the offset coefficient thus determined to the temperature value associated with each infrared image pixel so as to obtain a corrected electronic image, and to determine a maximum temperature value from the corrected electronic image and deduce therefrom an individual's body temperature;

Some preferred but non-limiting characteristics of the measurement system according to the third aspect are as follows, taken individually or in combination:
the first reference target, the second reference target, the first thermal sensor and the second thermal sensor are mounted on the archway;
the processing unit is also configured to apply a predetermined compensation coefficient to the temperature value of each infrared image pixel;
the measurement system also comprises a visible spectrum camera comprising a visible spectrum detection chip comprising a visible pixel matrix and being configured to generate a visible image comprising a plurality of visible image pixels, the visible spectrum camera being fixed onto the archway so that a field of view of the visible spectrum camera covers at least a portion of the passageway, the processing unit being also configured to identify the visible image pixels corresponding to at least a part of the individual's face, to match the visible image and the electronic image so as to identify the infrared image pixels corresponding to the visible image pixels identified, to determine the maximum temperature value associated with the infrared image pixels identified;
the infrared camera and the visible spectrum camera are mounted on an arm fixed onto the cross member and extending from an exit of the archway;
the measurement system also comprises a presence sensor configured to determine a presence of an individual in the passageway;
the first reference target and the second reference target are fixed onto one of the two side panels and the cross member;
the first reference target has a first reference temperature and the second reference target has a second reference temperature different from the first reference temperature;
the first reference temperature and the second reference temperature are comprised between 35° C. and 40° C.;
the measurement system also comprises a first heating element and a second heating element configured to maintain the first reference target and the second reference target at the first reference temperature and at the second reference temperature; respectively;
the measurement system also comprises a signalling unit configured to generate an alert when the maximum temperature value is greater than a predetermined threshold;
the processing unit is configured to identify the visible image pixels corresponding to the individual's eyes, preferably an inner corner of at least one eye of the individual; and/or
the measurement system also comprises a light, preferably flashing, fixed near the infrared camera so as to draw the gaze of an individual passing through the passageway.

According to a fourth aspect, the invention proposes a method for measuring an individual's body temperature by means of a measurement system according to the third aspect, comprising the following steps:

S1: producing an electronic image of a portion of the passageway in which an individual is located, the electronic image comprising a plurality of infrared image pixels representative of a temperature value received by a corresponding infrared pixel of a matrix of infrared pixels of an infrared camera;

S5: determining an instantaneous temperature of the first reference target and of the second reference target;

S6: determining in the electronic image the temperature value of the image pixels corresponding to the first reference target and to the second reference target;

S7: deducing therefrom a gain coefficient and an offset coefficient for the infrared camera;

S8: applying the gain coefficient and the offset coefficient to the temperature value associated with each image pixel so as to obtain a corrected electronic image; and

S9: determining a maximum temperature value associated with the identified infrared image pixels and deducing therefrom a body temperature of the individual.

Some preferred but non-limiting characteristics of the measurement method according to the fourth aspect are as follows, taken individually or in combination:
the method also comprises the following steps:

S2: producing a visible image of all or part of the portion of the passageway, the visible image comprising a plurality of visible image pixels;

S3: identifying the visible image pixels corresponding to at least a part of an individual's face, preferably at least one inner corner of the eyes;

S4: matching the visible image and the electronic image so as to identify the infrared image pixels corresponding to the visible image pixels identified in step S3; step S9 being implemented on the infrared image pixels identified in step S3;
steps S1 and S2 are simultaneous;
the method also comprises the application of a predetermined compensation coefficient to the temperature value of each image pixel or to the maximum temperature value determined in step S9;
the method also comprises, prior to step S1, a step S0 of determination of a presence of an individual in the archway, the steps S1 to S9 being implemented only when an individual is present in the passageway;
the method also comprises an alert generation step when the maximum temperature value is greater than a predetermined threshold; and/or
during step S3, the visible image pixels corresponding to the individual's eyes are identified, preferably visible image pixels corresponding to at least one inner corner of the eyes of the individual.

### DESCRIPTION OF THE FIGURES

Other characteristics, objectives and advantages of the invention will appear from the following description, which is purely illustrative and non-limiting and should be read with regard to the attached drawings, in which:

FIG. 1 schematically illustrates an example of embodiment of a measurement system conforming to one embodiment of the invention;

FIG. 2 is a partial face view of the measurement system of FIG. 1;

FIG. 3 is a partial rear view of the measurement system of FIG. 1;

FIG. 4 is a top view of the measurement system of FIG. 1 in which the light beams of the four photoelectric barriers are shown by a dashed line;

FIG. 5 is a side view of the measurement system of FIG. 1 in which examples of the field of views of the infrared camera and the visible spectrum camera have been shown;

FIG. 6 is an exploded view of an example of embodiment of a calibration module;

FIG. 7 is an exploded view of an example of embodiment of a housing comprising the infrared camera and the visible spectrum camera of a measurement system conforming to the invention;

FIG. 8 illustrates an example of an electronic image that can be obtained by the infrared camera of the measurement device of FIG. 1 in which an example of the calibration module has been illustrated schematically in detail;

FIGS. 9 and 10 are flow charts of the steps of a measurement method according to one embodiment of the invention;

FIG. 11 is a synoptic diagram of one example of embodiment of a measurement system conforming to the invention.

Throughout the figures, similar elements bear identical references.

### DETAILED DESCRIPTION OF THE INVENTION

In order to perform reliable, stable and contactless measurements of an individual's body temperature, the invention proposes a measurement system 10 for an individual's body temperature comprising:
an archway 11;
an infrared camera 12;
a visible spectrum camera 6;
optionally, a presence sensor 13 and/or a light 30, preferably flashing; and
a processing unit 15 configured to determine a body temperature of the individual.

### Archway 11

Archway 11 comprises two side panels 1 connected by a cross member 2 and together delimiting a passageway 9 for an individual. Side panel 1 and cross member 2 are connected mechanically so as to be of one piece.

Optionally, archway 11 can comprise an additional cross member 2, essentially parallel to cross member 2 and also connecting the two side panels 1.

Each side panel 1 has an inner face orientated toward passageway 9. More precisely, the inner face of the first panel faces the inner face of the second panel so as to laterally delimit the passageway. Side panels 1 also each have a first end, or entrance end, that delimit together an entrance 9a in the passageway for an individual, and a second end, or exit end, which is opposite the entrance end and defines exit 9b of passageway 9. Passageway 9 is therefore delimited by entrance 9a and exit 9b of archway 11, entrance 9a and exit 9b being defined by the direction of travel of an individual in archway 11.

Archway 11 also comprises a support, mounted on one of side panels 1 and cross member 2 and configured to receive infrared camera 12 and visible spectrum camera 6. In one embodiment, the support comprises an arm 7 comprising a first end mounted on archway 11, preferably at exit 9b of archway 11, and a second free end, opposite the first end and extending in the extension of archway 11. The infrared camera 12 and the visible spectrum camera 6 can be mounted, for example, on the second end of the arm 7 so as to be orientated toward the inside of the passageway 9.

The arm 7 thus makes it possible to easily mount and orient the cameras 6, 12 toward the inside of the passage. Moreover, this configuration makes it possible to move the infrared camera 12 and the visible spectrum camera 6 downstream of the archway 11 and therefore ensure that the images produced include images of the individual, when they are inside the passage. The fields of view 31, 32 of the cameras 6, 12 can actually cover the passageway 9 better than if they were mounted directly on the cross member 2 or the side panels 1.

Preferably, the arm 7 is mounted on the cross member 2 at the exit 9b of the archway 11 and extends perpendicularly to the cross member 2, downstream of the passageway 9.

The implementation of an archway 11 defining a passageway 9 for the individual advantageously makes it possible to ensure that only one individual at once is found in the field of view 31, 32 of the infrared camera 12 and the visible spectrum camera 6, and to thereby improve the precision of the measurements performed.

### Infrared Camera 12

The infrared camera 12 is configured to create an electronic image of the individual. For this purpose, it comprises an infrared detection chip 25 comprising a processor (or microprocessor) and a pixel matrix (hereinafter, “infrared” pixels), and an optical system 26, 27 configured to focus infrared energy on the infrared pixel matrix. Each infrared pixel of the matrix is configured to generate an electronic signal depending on an infrared energy entering by the optical system when creating the electronic image. This electric signal is transmitted to the processor of infrared detection chip 25 which converts into a corresponding temperature value. The processor generates an electronic image comprising a plurality of infrared images pixels, each infrared image pixel being representative of the temperature value received by a corresponding infrared pixel of the matrix. This electronic image can be, as appropriate, transmitted and displayed on a screen in the form of a color map representing the apparent temperature of the individual.

The infrared pixels of the infrared detection chip 25 are configured to detect an infrared energy having a wavelength greater than or equal to eight micrometers and less than or equal to fourteen micrometers. Each infrared pixel can have a maximum width greater than or equal to five micrometers and less than or equal to one hundred micrometers, depending on the total resolution of the infrared camera 12 sought. This resolution range makes it possible to precisely measure the temperature of zones of the individual with a small area, such as the internal corner of the eye.

The infrared detection chip 25 can be a microelectromechanical system (MEMS).

The infrared detection chip 25 is fixed onto a printed circuit 24. In one embodiment, the printed circuit 24 comprises a stud in which a through cavity is formed. The infrared detection chip 25 is then mounted in the cavity.

The optical system comprises one or more lenses 26 positioned in the optical axis of infrared camera 12 and configured to focus infrared energy on infrared detection chip 25. For example, the optical system can comprise a lens 26, mounted in the cavity in front of infrared detection chip 25. The optical system also comprises a diaphragm 27 positioned on the optical axis of lens 26 and configured to delimit an area for passage of an infrared beam. As applicable, diaphragm 27 can be mounted on or in front of the stud so as to close the cavity housing infrared detection chip 25 and insulate it from the external environment. The lens 26 and the diaphragm 27 can be made of germanium, for example, in order to allow infrared radiation to pass.

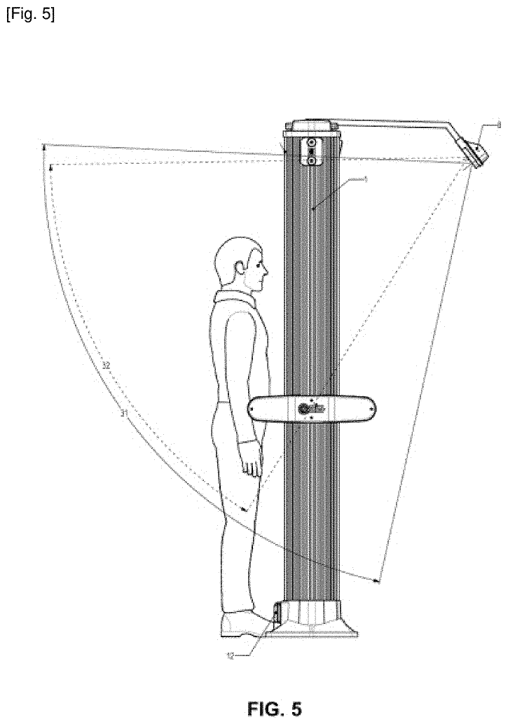

The infrared camera 12 is fixed onto the archway 11 so that its field of view 32 covers at least a portion of the passageway 9, preferably at least the upper portion of the passageway 9 that is intended to comprise the face and possibly the torso of the individual (see FIG. 5, for example). Upper portion here means the portion of the passageway 9 located next to the cross member 2 of the archway 11. The field of view 32 of the infrared camera 12 is defined by a vertical angle (φ) and a horizontal angle (θ) (vertical and horizontal being defined relative to the orientation of the archway 11 when it is in operation, i.e., when the archway 11 is set up on the ground or on a support and performs temperature measurements). In order to optimize the temperature measurement, the vertical angle (φ) and the horizontal angle (θ) are greater than or equal to 30° and less than or equal to 120° in order to limit the risk that the field of view 32 of the infrared camera 12 covers the environment and ensure that an individual's face is found in the field of view 32 of the infrared camera 12 when they pass through the passageway 9, regardless of their height.

### Visible Spectrum Camera 6

The visible spectrum camera 6 is configured to create a visible image of the individual. For this purpose, it comprises a visible spectrum detection chip 29 comprising a pixel matrix (hereinafter, “visible” pixels), and an optical system configured to focus visible electromagnetic radiation on the visible pixel matrix. Each visible pixel of the matrix is configured to generate an electronic signal depending on visible radiation entering by the optical system when creating the visible image. This electronic signal is transmitted to a processor (or microprocessor) of the visible spectrum detection chip 29, which converts it into a corresponding color. The processor generates a visible image comprising a plurality of visible image pixels, each visible image pixel being representative of the visible radiation received by a corresponding visible pixel of the matrix.

The visible pixels of visible spectrum detection chip 29 are configured to detect visible radiation having a wavelength greater than or equal to 0.4 micrometers and less than or equal to 0.7 micrometers. Each visible pixel can have a maximum width greater than or equal to one micrometer and less than or equal to thirty micrometers, depending on the total resolution of visible spectrum camera 6 sought. This resolution range makes it possible to obtain an image in which the zones of the individual with a small area, such as the internal corner of the eye, are identifiable with precision.

The visible spectrum detection chip 29 can be a microelectromechanical system (MEMS).

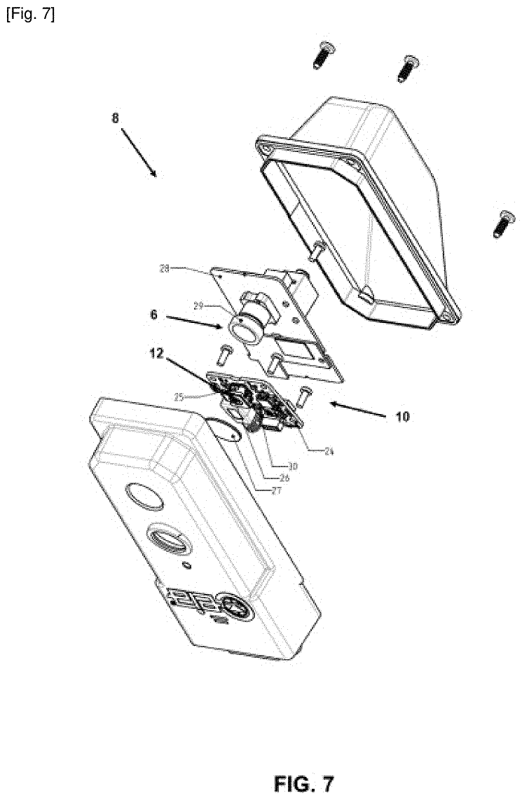

The visible spectrum detection chip 29 is fixed onto a printed circuit 28 as illustrated in FIG. 7. As applicable, visible spectrum detection chip 29 can be fixed onto the same printed circuit 24 as infrared detection chip 25.

The optical system of visible spectrum camera 6 is conventional and comprises, in a way known in and of itself, one or more lenses positioned on the optical axis of visible spectrum camera 6 and is configured to focus visible radiation on visible spectrum detection chip 29.

The visible spectrum camera 6 is fixed onto the archway 11 so that its field of view 31 covers at least the portion of the passageway 9 that is covered by the field of view 32 of the infrared camera 12. As applicable, the field of view 31 of the visible spectrum camera 6 can be larger than the field of view 32 of the infrared camera 12.

The printed circuits onto which the infrared camera 12 and the visible spectrum camera 6 are fixed are housed in a housing 8 comprising a base, onto which printed circuits 28, 24 are fixed and a cover 23 is attached and fixed to the base. The cover 23 comprises a first opening, positioned facing the infrared camera 12 and a second opening positioned facing the visible spectrum camera 6. As a variant, a single opening positioned facing both cameras 6, 12 can be created in cover 23.

The diaphragm 27 can especially be mounted between the infrared camera 12 and the cover 23.

In an embodiment, the measurement system 10 comprises only one infrared camera 12, 6 (or two infrared cameras 12, 6), the infrared camera 6 also implementing the functions performed by the visible spectrum camera 6.

### Processing Unit 15

The processing unit 15 can notably comprise a computer of the processor, microprocessor, microcontroller, etc. type, configured to execute instructions and control the processor of the infrared detection chip 25, the visible spectrum camera 6 and, optionally, a presence sensor 13 and/or at least one signalling unit 33 (detailed below).

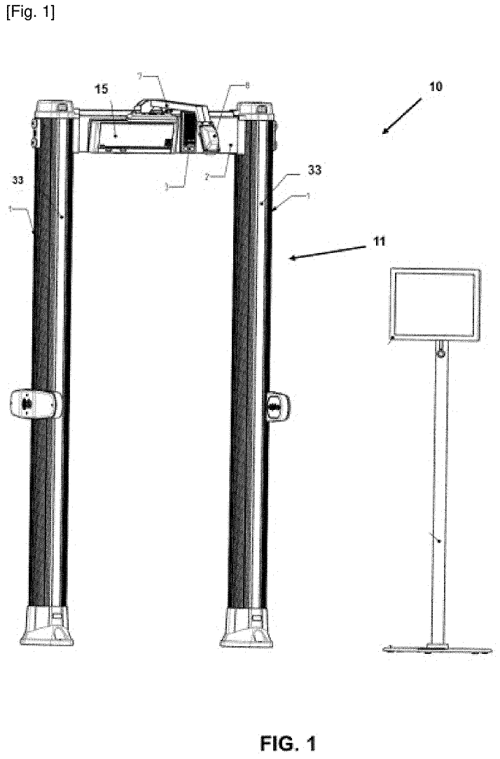

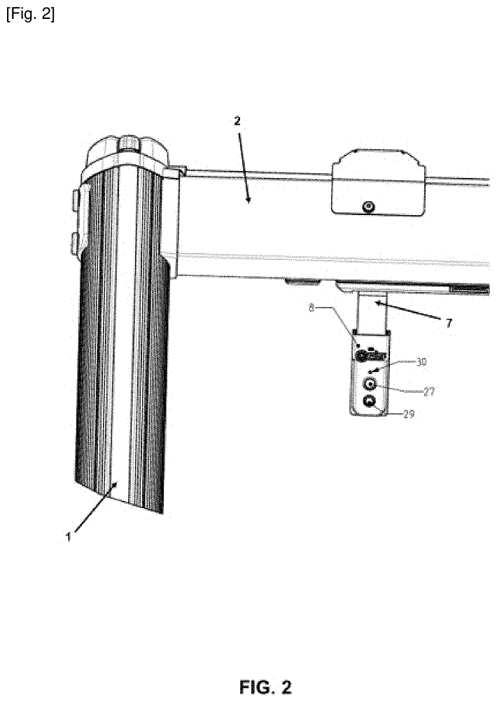

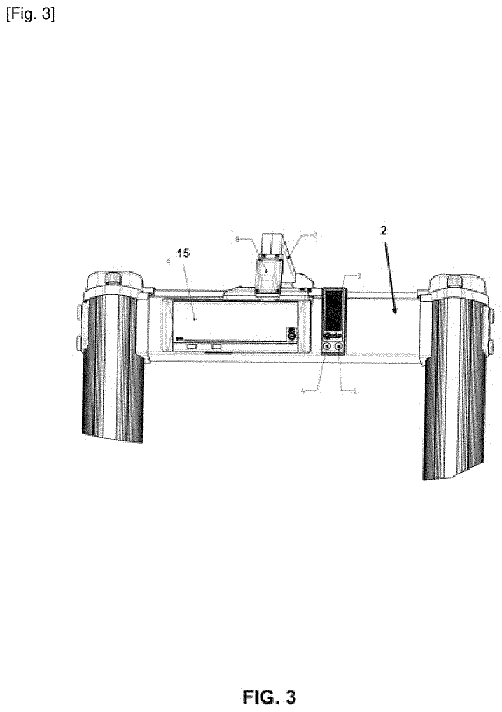

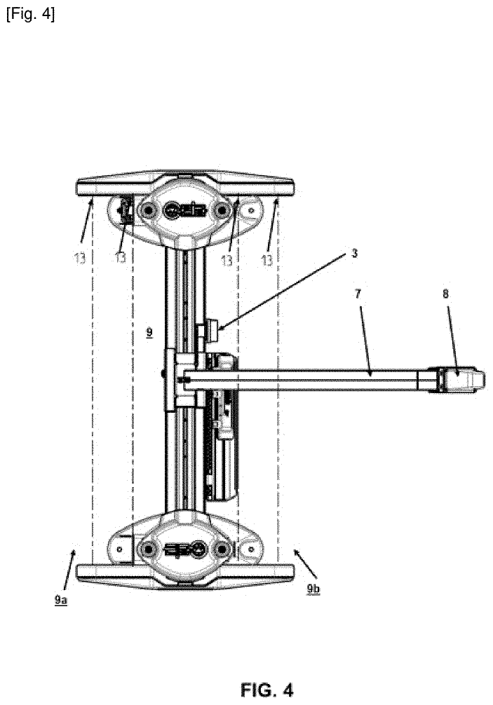

In an embodiment, the processing unit 15 is mounted on the same printed circuit as the infrared detection chip 25. The processing unit 15 can notably integrate the processor of the infrared detection chip 25. As a variant, the processing unit 15 and the processor of the infrared camera 12 can be separate, in which case the processing unit 15 can be housed in the archway 11 at a distance in a separate console of the archway 11 (see FIG. 1, for example) or in a network.

The processing unit 15 is configured to determine the body temperature of an individual passing through the passageway 9 from the electronic image obtained by the infrared camera 12 and, as applicable, from the visible image obtained by the visible spectrum camera 6.

In the case where the system 10 comprises a visible spectrum camera 6, the processing unit 15 is connected to the visible spectrum camera 6 and configured to receive the visible image and identify in this visible image the visible image pixels that are representative of at least a part of an individual's face. In one embodiment, the processing unit 15 preferably identifies only a part of the face, typically the eyes or even an inner corner of the eyes. Indeed, the area presenting the highest temperature in a face generally corresponds to the inner corner of the eye.

The processing unit 15 is further connected to the infrared camera 12 and configured to receive the electronic image and match it to the electronic image so as to identify in the electronic image the infrared image pixels that correspond to the visible image pixels representative of the face, or, as applicable, to the eyes and/or the inner corner of one eye or both eyes. This correspondence makes it possible, in particular, to ensure that the temperature values calculated from the electronic image properly correspond to the individual's temperature values, and not to their environment. The particular choice of the eyes, and more particularly still the inner corner of the eyes also makes it possible to limit disruptions due to the environment, by ensuring that the value of the temperature measured is close to the individual's body temperature.

Finally, regardless of the configuration (with or without visible spectrum camera 6), processing unit 15 is configured to determine a maximum temperature value Tmax associated with the infrared image pixels corresponding to the face, eyes and/or inner corner of the eyes, and to deduce therefrom the individual's body temperature.

For comparison purposes, conventional measurement systems, and especially portable systems (with or without contact), do not allow performing a measurement in the corner of the eye, either because they are designed to be contacted with the forehead or inside the individual's ear, or because it would not be possible for an operator to check an individual's temperature at the entrance to a limited access area by taking a temperature at the eye. On the contrary, since the invention creates an electronic image of the individual, it is possible to determine in the image the point having the highest temperature, which generally corresponds to the inner corner of the eye, without contact and without requiring the intervention of an operator.

When the system 10 only comprises an infrared camera 12, 6, the processing unit is configured to directly determine in the electronic image the infrared electronic pixels the infrared image pixels that correspond to the visible image pixels representative of the face, or, as applicable, to the eyes and/or the inner corner of one eye or both eyes.

Thus, only the area of the face, and preferably only the area of the face comprising the eyes and/or the inner corner of the individual's eyes, is actually measured and it is the maximum temperature Tmax in this limited area that is compared to a predetermined threshold

Tthreshold. The use of measurement system 10 therefore makes it possible to ensure that a temperature value is obtained that is very close or even equal to the individual's body temperature.

### Presence Sensor 13

In order to limit external disruptions when measuring an individual's temperature, measurement system 10 can also comprise a presence sensor 13 configured to determine the presence of an individual inside the passageway 9 of the archway 11. The processing unit 15 is then configured so as to generate an electronic image and a visible image only when the presence sensor 13 detects an object (presumably an individual) inside the passageway 9.

The Applicant realizes that the environment in which the system is positioned generates infrared energy that is likely to disrupt temperature measurements. For example, it can be noted that light emitting diode (LED) or luminescent tube lighting have a temperature of around 40° C., which is a temperature that would be symptomatic of a fever and is therefore able to disrupt temperature measurement by the system. By generating the electronic image only when an individual is found inside the passageway 9, it is therefore possible to ensure that an individual occupies the field of view 31, 32 of the infrared camera 12 and the visible spectrum camera 6 when creating the electronic and visible images and that the temperature measurement is not disrupted by external elements.

The presence sensor 13 can be fixed onto the archway 11, for example on one of the panels or the cross member 2. For example, the presence sensor 13 can comprise one or more photoelectric barriers fixed onto the inner faces opposite the panels. Preferably, the system comprises at least one photoelectric barrier positioned at the entrance 9a of the archway 11 and one photoelectric barrier positioned at the exit 9b of the archway 11 (and optionally one or more photoelectric barriers distributed between the two), the processing unit 15 being thus configured to generate an electronic image and a visible image between the moment when the photoelectric barrier located at the entrance 9a and when the photoelectric barrier at the exit 9b each detect the presence of an individual.

In a way known in and of itself, the photoelectric barriers each comprise a light beam emitter, mounted on one of the inner faces of side panels 1, and a light beam receiver, mounted on the other inner face. As a variant, the emitter and the receiver can be fixed onto the same side panel 1, the photoelectric barrier then comprising a reflector fixed onto the opposite inner face and configured to reflect the light signal emitted by the emitter onto the reflector. During the passage of an individual, the reception of the light beam by the receiver is interrupted: evaluation electronics (of the microprocessor type) then send a defined electrical signal to the processing unit 15 signalling the passage of an individual. When the photoelectric barrier is placed at the entrance 9a of the archway 11, the central unit deduces therefrom that an individual is present in the passageway 9. When the photoelectric barrier is placed at the exit 9b of the archway 11, the central unit deduces therefrom that an individual has left the passageway 9.

### Light 30

Optionally, the measurement system 10 can also comprise a light 30, preferably flashing, positioned near the infrared camera 12 in order to draw the gaze of the individual when the electronic and visible images are being created. For example, the light 30 can be mounted on the housing 8 near the infrared camera 12.

In one embodiment, the light 30 can comprise a light-emitting diode (LED).

The light 30 can be lit, and, as applicable, flashing, continuously. As a variant, the processing unit 15 can be connected to the light 30 so as to light it, and, as applicable, make it flash, only when the electronic image and the visible image have to be created. Typically, when the measurement system 10 comprises a presence sensor 13, the processing unit 15 can turn on the light 30, and, as applicable, make it flash, when an individual is detected by the presence sensor 13.

### Signalling Unit 33

In one embodiment, the measurement system 10 can also comprise at least one signalling unit 33 configured to generate an optical alert (light signal) and/or sonic alert (acoustic signal) when the maximum temperature value Tmax exceeds a predetermined threshold.

Optionally, the signalling unit 33 can also be configured to generate a signal when the maximum temperature value Tmax is less than or equal to the predetermined threshold Tthreshold in order to signal to an operator that a measurement has been done but the individual's body temperature is below the threshold. For example, the signalling unit 33 can comprise a green light and a red light. The processing unit 15 can then send instructions for lighting the red light when maximum temperature Tmax is greater than the predetermined threshold temperature Tthreshold and for the green light when it is less than or equal to this predetermined threshold.

### Measurement Method

An individual's body temperature can be measured using the measurement system 10 conforming to the following steps.

During an initial step S0, the presence of an individual in the archway 11 and an individual is determined by the presence sensor 13. For this, the processing unit 15 interrogates the presence sensor 13, such as a photoelectric barrier.

In the case of a presence sensor comprising photoelectric barriers, when the barrier located at the entrance 9a (respectively at the exit) of the archway 11 sends a presence signal to the processing unit 15, this unit deduces therefrom that an individual has entered (respectively left) the passageway 9. The processing unit 15 thus triggers measurement of the individual's body temperature between the receipt of the presence signal from the barrier located at the entrance 9a and the receipt of the presence signal from the barrier located at the exit 9b.

However, if the photoelectric barriers, and especially the barrier at the entrance 9a, do not send the presence signal, the processing unit 15 deduces therefrom that no individual is present in the passageway 9 and does not trigger the temperature measurement.

During a step S1, the processing unit 15 sends instructions to the infrared camera 12 to create an electronic image. As indicated above, the infrared camera 12 is oriented, as appropriate, by the arm 7 so that its field of view 32 covers all or part of the passageway 9 including at least an upper portion.

Since the electronic image is created between the detections performed by the barriers at the entrance 9a and the exit 9b of the archway 11, it necessarily comprises the individual. Moreover, since the infrared camera 12 is mounted onto the archway 11 so that its field of view 32 covers at upper least the upper portion of the passageway 9, the face and possibly the torso of the individual is found in the field of view 32 of the infrared camera 12.

During a step S2, the processing unit 15 sends instructions to the visible spectrum camera 6 to create a visible image of all or part of the portion of the passageway 9.

In an embodiment, the field of view 31 of the visible spectrum camera 6 and the field of view 32 of the infrared camera 12 essentially cover the same portion of the passageway 9 and in any event the upper portion thereof so that the visible image and the electronic image both cover the face and torso of the individual.

Optionally, the field of view 32 of the infrared camera 12 and/or the field of view 31 of the visible spectrum camera 6 can cover the entire height of the passageway 9.

Preferably, steps S1 and S2 are simultaneous, in order to facilitate matching the visible and infrared images. Moreover, in one embodiment, the light 30 can be lit by the processing unit 15 (or be lit continuously), as applicable in a flashing manner, in order to draw the gaze of the individual when the electronic and visible images are created during steps S1 and S2.

During a step S3, the processing unit 15 identifies in the visible image the visible image pixels that correspond to the individual's face, the individual's eyes and/or to the inner corners of the eyes. Preferably, the processing unit 15 identifies the visible image pixels corresponding to at least one inner corner of the eyes.

For this, the processing unit 15 detects the face (respectively, the eyes and/or at least one inner corner of the eyes) according to the method of Viola and Jones (or integral image), which is a supervised learning method using a Haar cascade classifier. For further detail, refer to the article by Paul Viola and Michael Jones, “Rapid Object Detection using a Boosted Cascade of Simple Features”, 2001 IEEE Computer Society Conference on Computer Vision and Pattern Recognition, for further details on this method.

Other methods can be used for the performance of step S3, such as deep learning methods using a semantic segmentation classifier. One can also refer to the article by Alex Krizhevsky, Ilya Stuskever and Geoffroy E. Hinton, “ImageNet Classification with Deep Convolutional Neural Networks” or to the article by Vijay Badrinarayanan, Alex Kendall and Roberto Cipolla, “SegNet: A Deep Convolutional Encoder-Decoder Architecture for Image Segmentation”, for further details on the use of semantic segmentation classifiers. These methods can be used, as applicable, and combined to increase the detection speed and the precision of the characteristics sought.

During a step S4, the processing unit 15 matches the visible image and the electronic image so as to identify the infrared image pixels corresponding to the visible image pixels identified in step S3.

The visible and infrared image pixels can be matched by transposition of the image pixel coordinates of the visible image to the electronic image by taking into account the positions and orientations of the visible spectrum camera 6 and the infrared camera 12, in a way known to the skilled person. For example, this can be done by learning the characteristics of the cameras 6, 12 automatically (parameters intrinsic to the cameras 6, 12 such as focal length and distortion, and extrinsic parameters such as position and orientation). This learning takes place once and for all, typically during the installation and calibration of the measuring system 10. For example, the transposition can be done by implementing an affine application of the visible image in the electronic image. For this purpose, the affine application coefficients are determined when the measurement system 10 is calibrated. To do so, visual references can be positioned in space at known positions relative to, then visible and infrared images of these visual references are made with the cameras 6, 12. Knowing the three-dimensional position of the visual references relative to the two cameras 6, 12, it is possible to determine the affine application coefficients for the visible image in the electronic image.

In a step S9, the processing unit 15 determines the maximum temperature value Tmax associated with the infrared image pixels identified at step S4 and deduces therefrom the individual's body temperature.

### Calibration of Infrared Measurements

In one embodiment, to correct the electronic image obtained by infrared camera 12 and ensure the reliability of the body temperature measurement, the measurement system 10 also comprises:
a support 16;
a first reference target 4 and a second reference target 5 positioned in field of view 32 of infrared camera 12; and
a first thermal sensor 20 and a second thermal sensor 22 configured to measure an instantaneous temperature of first reference target 4 and second reference target 5.

Furthermore, the processing unit 15 is also configured to determine a gain coefficient and an offset coefficient for the infrared camera 12 and thus correct the electronic image obtained by the infrared camera 12 in view of determining in step S9 the maximum temperature value Tmax associated with the infrared image pixels identified in step S4.

In the following, the invention will be described generally in the case where the processing unit 15 performs the calibration of the infrared measurements. However, this is not limiting; this calibration can be done by any processor or microprocessor, such as a dedicated microcontroller which is controlled by the processing unit 15 or by an external local computer or a remote networked computer.

### First Reference Target 4 and Second Reference Target 5

The first reference target 4 has a first reference temperature and the second reference target 5 has a second reference temperature and has for a function making it possible to calibrate the infrared camera 12 during the creation of each electronic image. The first reference temperature and the second reference temperature are preferably different. The first reference target 4 and the second reference target 5 can, for example, each comprise a surface, heated to the corresponding reference temperature. The first reference target 4 and the second reference target 5 can, for example, each comprise a surface, which can be essentially flat, heated to the corresponding reference temperature.

In order to ensure an optimal correction of the electronic image created by the infrared camera 12, the first and the second temperatures are close to the body temperature at which it is usually believed that an individual presents symptoms of fever, typically 37.5° C. The first and the second reference temperature can be comprised, for example, between 35° C. and 40° C. In one embodiment, the first reference temperature is equal to 36° C. and the second reference temperature is equal to 39° C.

The first reference target 4 and the second reference target 5 are each positioned in the field of view 32 of the infrared camera 12 so that the electronic image comprises image pixels representative of their respective temperature value. Preferably, the first reference target 4 and the second reference target 5 are fixed relative to the infrared camera 12 and are placed in the field of view 32 of the infrared camera 12.

The first reference target 4 and the second reference target 5 can, for example, be fixed onto the archway 11. In one embodiment, the first reference target 4 and the second reference target 5 are fixed onto the cross member 2 of the archway 11, in the field of view 32 of the infrared camera 12. This configuration can notably be envisaged when the infrared camera 12 is mounted on an arm 7 so as to extend downstream from the passageway 9. Advantageously, the fixation of the first and second reference targets 4, 5 on the cross member 2 makes it possible to ensure that the reference targets 4, 5 are fixed relative to the infrared camera 12 and do not risk being masked by the individual when they pass through the passageway.

As applicable, the first reference target 4 and the second reference target 5 can be housed in the same housing 3.

The position and area of the first reference target 4 and the second reference target 5 relative to the infrared camera 12 are chosen so that the first reference target 4 and the second reference target 5 each extend in the field of view 32 for at least one pixel of the infrared detection chip 25, for example for a sub-matrix comprising 3×3 pixels. To do so, the first and the second reference target 4, 5 can be positioned at a distance between 20 cm and 1.5 meters from the infrared detection chip 25 and have an area comprised between 25 mm² and 400 cm².

In one variant of embodiment, the first reference target 4 and the second reference target 5 can be fixed onto the inner surface of one and/or the other of side panels 1 of the archway 11, preferably near the cross member 2.

Optionally, in order to protect the first and second reference targets 4, 5, the system can also comprise a diaphragm 17, 18 positioned in front of each reference target 4, 5 in order to protect them from the environment and prevent any disruptions (such as the presence of an air current or any element likely to change the temperature of the reference targets 4, 5). As applicable, the diaphragms 17, 18 can be mounted between the first reference target 4 and the second reference target 5, respectively, and a cover of the housing 3.

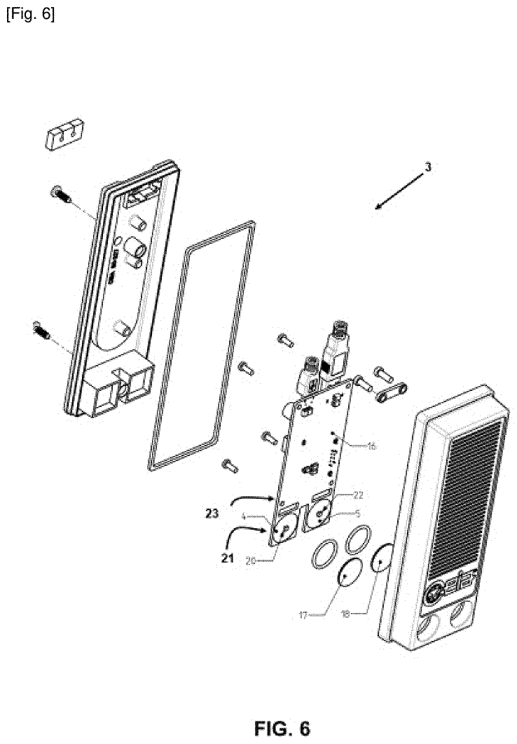

The measurement system 10 also comprises a first heating element 21 and a second heating element 23 configured to maintain the first reference target 4 and the second reference target 5 at the first reference temperature and the second reference temperature; respectively. The first and second heating elements 21, 23 can, for example, each comprise a resistor connected to the first and second reference targets 4, 5, respectively. In one embodiment, the first and second heating elements 21, 23 are mounted on the face opposite the infrared camera 12 (which corresponds to the face opposite the first and second reference targets 4, 5) so as not to be seen by the infrared camera 12 (see FIG. 6).

As applicable, the first and second heating elements 21, 23 can be housed in the same housing 3 as the first and second reference targets 4, 5.

### First and Second Thermal Sensors 20, 22

The first and second thermal sensors 20, 22 are configured to measure an instantaneous temperature of the first reference target 4 and the second reference target 5, respectively. Preferably, the first and second thermal sensors 20, 22 are in contact (direct or indirect) with the first or second reference targets 4, 5, respectively. The first and second thermal sensors 20, 22 are preferably in one piece with the first or second reference targets 4, 5, respectively. It will be noted that, preferably, the first and second heat sensors 20, 22 are preferably positioned so as not to form an obstacle between the individual and the infrared camera 12.

Preferably, the first and second thermal sensors 20, 18 have a measurement precision less than or equal to 0.1° C. in order to provide a very precise value of the instantaneous temperature of each reference target 4, 5. As we will see below, it is this instantaneous temperature value of the first and second reference targets 4, 5 that is then used by the processing unit 15 and not the value of the first and second reference temperatures programmed, to correct the electronic image generated by the infrared camera 12.

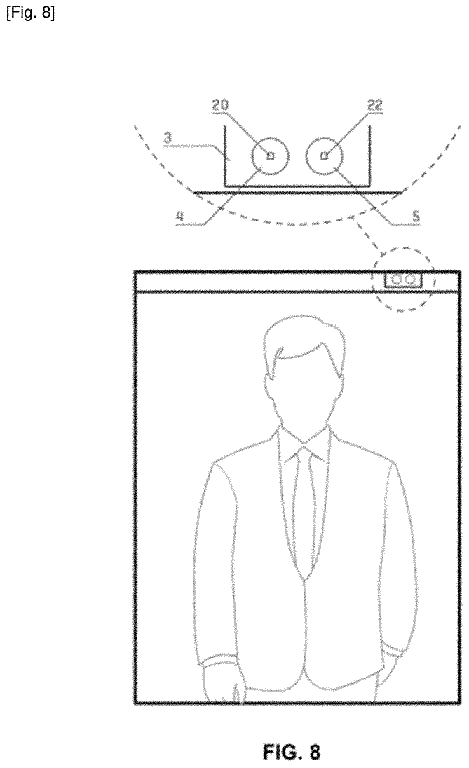

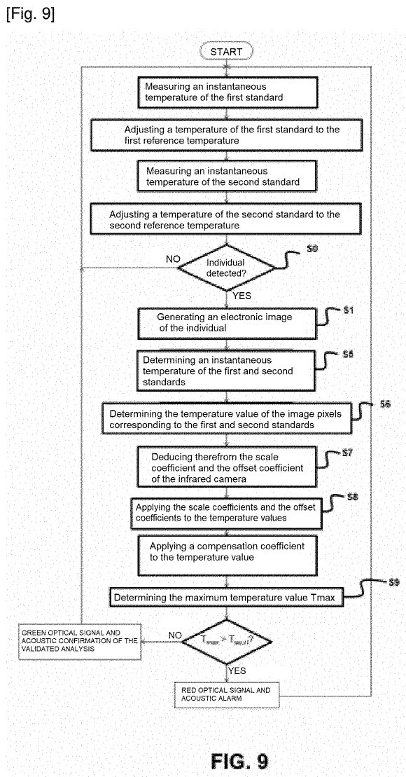

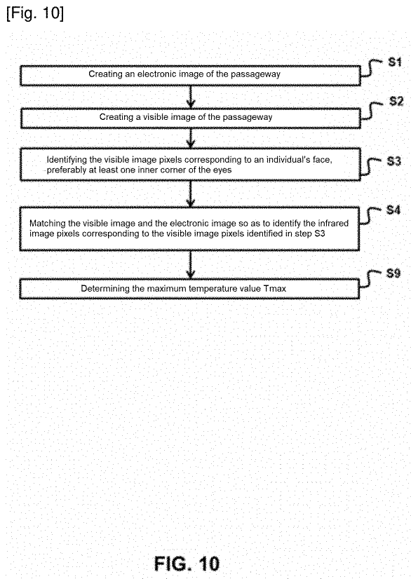

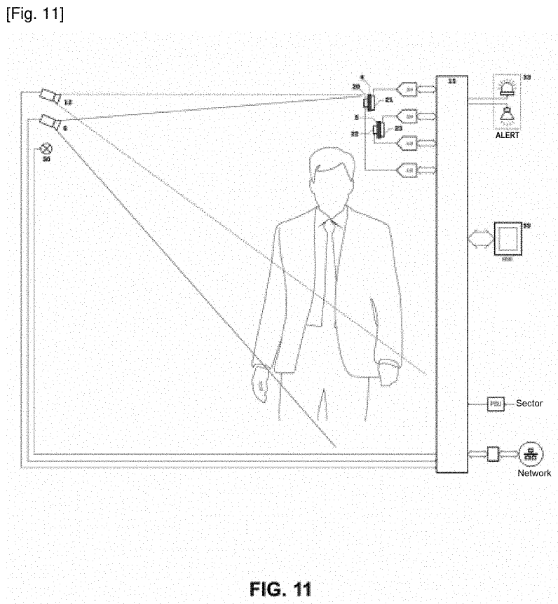

In one embodiment, the thermal sensors 20, 22 are connected to corresponding reference targets 4, 5 by means of a conductive track. However, the Applicant has noticed that, in operation, the temperature of this conductive track was essentially equal (within 0.1° C.) to the temperature of the heated surface of the corresponding reference target 4, 5. Consequently, in one embodiment, the conductive track that connects the first thermal sensor 20 to the heated surface of the first reference target 4 and the second thermal sensor to the heated surface of the second reference target 5 can be considered as being part of the reference targets 4, 5, respectively, by the processing unit 15. In this embodiment, the first and second thermal sensors 20, 22 can therefore be mounted in the immediate vicinity of the first reference target 4 and the second reference target 5. For example, the first heat sensor 20 can be fixed at the center of the heated surface of the first reference target 4 and the second heat sensor 22 can be fixed at the center of the heated surface of the second reference target 5 (see FIG. 8, for example). The first and second thermal sensors 20, 22 can comprise a semiconductor chip, for example. The reference targets 4, 5 are then encased by the upper part of the semiconductor chip, at the edge of the reading area.

When the support 16 comprises an additional printed circuit, the thermal sensors can be mounted on this additional printed circuit.

As applicable, the first and second heating elements 21, 23 can be controlled by the processing unit 15 depending on the instantaneous temperature value of the first and second reference targets 4, 5 which is measured by the first and second thermal sensors 20, 22.

In order to correct the electronic image of the infrared camera 12, the processing unit 15 is also configured to determine a gain coefficient and an offset coefficient from the respective instantaneous temperatures of the first reference target 4 and the second reference target 5 and the temperature values of the first reference target 4 and the second reference target 5 in the electronic image and to apply the gain coefficient and the offset coefficient determined to the temperature value associated with each infrared image pixel so as to obtain a corrected electronic image.

Thus, unlike conventional measurement devices, since the measurement system 10 creates an electronic image of the individual and determines the temperature value at each pixel of the image, it is not satisfied with taking a measurement at a random point on the individual's face and thus limits the risks of the measurement being disrupted by the environment.

The processing unit 15 is therefore configured to execute the instructions and control the first and second thermal sensors 20, 22 and the first and second heating elements 21 and 23.

### Method for Calibration of Infrared Measurements

The temperature values associated with the infrared image pixels identified in step S4 can then be calibrated using the measurement system 10 according to the following steps.

During step S5, the instantaneous temperature of the first reference target 4 and the second reference target 5 is determined by the first thermal sensor 20 and the second thermal sensor 22, respectively.

Preferably, steps S1 and S5 are simultaneous. Simultaneous here means that steps S1 and S5 are conducted at the same time, or with a time lag at most equal to the time necessary for the temperature of the first reference target 4 and the second reference target 5 to change by 0.1°.

During a step S6, the processing unit 15 determines in the electronic image obtained in step S1 the temperature value of each infrared image pixel corresponding to the first reference target 4 and the second reference target 5. The infrared image pixels corresponding to the reference targets 4, 5 can be identified in the electronic image insofar as the spatial position of the first and second reference targets 4, 5 relative to the infrared camera 12 is known and fixed.

In one embodiment, notably when the thermal sensors comprise a semiconductor chip, the processing unit 15 can more particularly determine the temperature value of each infrared image pixel corresponding to the conductive track connecting the first reference target 4 and the second reference target 5 to the first thermal sensor 20 and the second thermal sensor 22. As we have seen above, these conductive tracks actually have a temperature essentially equal to that of the corresponding reference targets 4, 5 and are therefore considered to be part of the first reference target 4 and the second reference target 5, respectively. The area where the thermal sensors 20, 22 and the heated surface of the reference targets 4, 5 are superimposed makes it possible to significantly reduce errors caused by thermal drops. As a variant, the processing unit 15 can determine the temperature value associated with the heated surface of the reference targets 4, 5. When several image pixels correspond to the first reference target 4 and the second reference target 5 in the electronic image, the processing unit 15 can notably choose the mean temperature value among the submatrix of image pixels as the instantaneous value of the corresponding reference target 4, 5.

During a step S7, the processing unit 15 deduces from the temperature values determined in S6 and the instantaneous temperature of the first and second reference targets 4 and 5 measured at step S5 a gain coefficient k₁ and an offset coefficient k₂ of the infrared camera 12. A gain coefficient k₁ corresponds to a deviation in amplitude of the infrared camera 12 while an offset coefficient k₂ corresponds to an error having a constant value corresponding to an offset of the values measured on the y-axis of the output voltage of the infrared detection chip 25.

More precisely, the instantaneous temperature T_{i_1} (respectively, T_{i_2}) of the first reference target 4 (respectively of the second reference target 5) is equal to the gain coefficient K₁ multiplied by the temperature value T_{IR_1} (respectively, T_{IR_2}) determined in step S3 for the first reference target 4 (respectively for the second reference target 5) and for the offset coefficient k₂:

T_{i_1}=T_{IR_1}*k₁+k₂

T_{i_2}=T_{IR_2}*k₁+k₂

The instantaneous temperature T_{i_1}, T_{i_2} of the first reference target 4 and the second reference target 5 are independent from the deviation of the infrared camera 12, being measured by the first and the second thermal sensors 20, 22. Furthermore, the deviation of the infrared camera 12 is the same for the temperature values T_{IR_1} and T_{IR_2} determined at step S3. As a result, k₁ and k₂ are identical in these two equations. Thus, the resolution of these equations permitted by the presence of the two reference targets 4, 5 and the determination of their instantaneous temperature T_{i_1} and T_{i_2} by the associated thermal sensors 20, 22 makes it possible to determine the value of the gain coefficient k₁ and the offset coefficient k₂ of the infrared camera 12.

During a step S5, the processing unit 15 applies the gain coefficient k₁ and the offset coefficient K₂ determined in step S4 to the temperature value associated with each image pixel so as to obtain a corrected electronic image.

Thus, for any infrared image pixel i of the electronic image generated in step S3, the processing unit 15 applies the gain coefficient k₁ and the offset coefficient k₂ to the temperature value T_{IR_i} associated with this infrared image pixel i so as to obtain, for each image pixel i, the corrected temperature value T_{corr_i}; and deduce therefrom the corrected electronic image that comprises the corrected infrared image pixels:

T_{corr_i}=k₁*+T_{IR_i}+k₂

During step S9, the processing unit 15 therefore determines the maximum temperature value Tmax in the corrected electronic image and compares this maximum temperature value Tmax with a predetermined threshold Tthreshold. Since the body temperature from which an individual is usually considered to present symptoms of fever is generally 37.5° C., the predetermined threshold Tthreshold can be equal to 37.5° C., for example.

When the maximum temperature Tmax is greater than or equal to the predetermined threshold temperature Tthreshold, the processing unit 15 sends instructions to the signalling unit 33 in order to generate an alert (typically a visual and/or acoustic alert).

In one embodiment, the measurement method also comprises a step S8 during which the processing unit 15 applies a predetermined compensation coefficient K₃ to the temperature value of each infrared image pixel in order to compensate for a difference of emissivity between the first and second reference targets 4, 5 and human skin and thereby obtain a corrected temperature value closer to the actual body temperature of the individual:

T_{final_i}=T_{corr_i}*k₃

It is then this corrected and compensated temperature value T_{final_i} which is used in step S9 to determine the maximum temperature value Tmax, and not the corrected value T_{corr_i}.

In one variant of embodiment, the compensation coefficient can be applied only to the maximum temperature value Tmax determined in step S9. Step S8 then takes place after step S9.

The compensation coefficient k₃ is a fixed and predetermined coefficient, which does not depend on any thermal offset or environmental measurement. In return, this coefficient depends on the emissivity of human skin and the emissivity of the first and second reference targets 4, 5, and, more particularly, the material making up their heated surface, or, as applicable, the conductive track which is connected to it. This is why it may be preferable to create the first and second reference target 4, 5 in the same constituent material, only their respective reference temperature being different.

In one embodiment, steps S1 to S9 can be implemented continuously according to the same interrogation period (which can be comprised between ten milliseconds and five hundred milliseconds of interrogation), independently of the detection of an individual in the passageway 9. In this case, the processing unit 15 only sends instructions for generating an alert to the signalling unit 33 if the presence sensor 13 detects an individual in the passageway 9.

Moreover, steps S1 to S9 are repeated as long as the individual is in the passageway 9, typically as long as the photoelectric barrier located at the exit 9b of the passageway 9 has not detected the exit of the individual. The period for repeating these steps can notably be equal to the interrogation period of the presence sensor 13 by the processing unit 15.

Furthermore, it will be noted that at any time during the operation of the system (and therefore during the interrogation of the presence sensor 13 by the processing unit 15), the first and second heating elements 21, 23 maintain the first reference target 4 and the second reference target 5 at the first reference temperature and the second reference temperature, respectively, so as to guarantee that the instantaneous temperature of said elements is close to their respective reference temperature during steps S1 and S5.

In a way known in and of itself, the measurement 10 also comprises a kiosk consisting of a base and a screen, connected to the system processing unit, for example by an Ethernet interface.

1. A temperature measurement system comprising:
an archway comprising two side panels connected by a cross member and together delimiting a passageway for an individual;
an infrared camera comprising an infrared detection chip comprising an infrared pixel matrix and being configured to convert an infrared radiation received by each infrared pixel into a corresponding temperature value and to generate an electronic image comprising a plurality of infrared image pixels, each infrared image pixel being representative of the temperature value received by a corresponding infrared pixel, the infrared camera being fixed onto the archway so that a field of view of the infrared camera covers all or part of the passageway;
a calibration module comprising:
a first reference target and a second reference target positioned in the field of view of the infrared camera so that the electronic image comprises the infrared image pixels representative of the temperature value of the first reference target and the second reference target; and
a first thermal sensor configured to measure an instantaneous temperature of the first reference target and a second thermal sensor configured to measure an instantaneous temperature of the second reference target; and
a processing unit configured to:
identify the infrared pixels corresponding to at least a part of an individual's face and to determine a maximum temperature value associated with the identified infrared pixels and to deduce therefrom a body temperature of the individual;
determine a gain coefficient and an offset coefficient from the respective instantaneous temperatures of the first reference target and the second reference target and temperature values of the first reference target and the second reference target in the electronic image;
apply the gain coefficient and the offset coefficient thus determined to the temperature value associated with each infrared image pixel so as to obtain a corrected electronic image; and
determine the maximum temperature value from the corrected electronic image.

2. (canceled)

3. The temperature measurement system of claim 1, further comprising a visible spectrum camera comprising a visible spectrum detection chip comprising a visible pixel matrix and being configured to generate a visible image comprising a plurality of visible image pixels, the visible spectrum camera being fixed onto the archway so that a field of view of the visible spectrum camera covers at least a portion of the passageway, the processing unit being configured to match the visible image and the electronic image to identify the infrared image pixels corresponding to the visible image pixels identified.

4. The temperature measurement system of claim 1, wherein the processing unit is also configured to apply a predetermined compensation coefficient to the temperature value of each infrared image pixel.

5. The temperature measurement system of claim 1, wherein the first and the second thermal sensors are connected to the first and second reference targets, respectively, by means of a first and a second conductive track, the processing unit being configured to determine an instantaneous temperature of the first and the second conductive tracks and deduce therefrom the instantaneous temperature of the first and second reference targets respectively.

6. The temperature measurement system of claim 5, wherein the first and second thermal sensors each comprise a semiconductor chip connected to the first and second reference targets, respectively, by means of the first and second conductive tracks.

7. The temperature measurement system of claim 6, wherein the semiconductor chip of the first and second thermal sensors is fixed at a center of a heated surface of the first reference target and the second reference target, respectively.

8. The temperature measurement system of claim 1, wherein the first reference target and the second reference target are fixed onto one of the two side panels and the cross member.

9. The temperature measurement system of claim 1, also comprising a presence sensor configured to determine a presence of an individual in the passageway, the processing unit being configured to generate the electronic image and the visible image only when the presence sensor detects an object inside the passageway.

10. The temperature measurement system of claim 1, wherein the infrared camera and the visible spectrum camera are mounted on an arm fixed onto the cross member and extending from an exit of the archway.

11. The temperature measurement system of claim 1, further comprising a light adjacent to the infrared camera and configured to draw a gaze of an individual passing through the passageway.

12. The temperature measurement system of claim 1, wherein the field of view of the infrared camera has a vertical angle and a horizontal angle greater than or equal to 30° and less than or equal to 120°.

13. A temperature measurement method comprising:
S1: producing an electronic image of a portion of a predetermined passageway in which an individual is located, the electronic image comprising a plurality of infrared image pixels representative of a temperature value received by a corresponding infrared pixel of a matrix of infrared pixels of an infrared camera, a first reference target and a second reference target being positioned in a field of view of the infrared camera so that the electronic image comprises infrared image pixels representative of a temperature value of the first reference target and the second reference target;
S4: identifying the infrared pixels corresponding to at least a part of the individual's face;
S5: determining an instantaneous temperature of the first reference target by means of a first thermal sensor and of the second reference target by means of a second thermal sensor;
S6: determining in the electronic image the temperature value of the infrared image pixels corresponding to the first reference target and to the second reference target;
S7: deducing therefrom a gain coefficient and an offset coefficient for the infrared camera;
S8: applying the gain coefficient and the offset coefficient to the temperature value associated infrared image pixels to obtain a corrected electronic image; and
S9: determining a maximum temperature value associated with the identified infrared pixels and deducing therefrom a body temperature of the individual.

14. The temperature measurement method of claim 13, also comprising the following steps, prior to step S9:
S2: producing a visible image of all or part of the predetermined passageway, the visible image comprising a plurality of visible image pixels; and
S3: identifying the visible image pixels corresponding to at least a part of an individual's face;
wherein in step S4, the infrared pixels are identified by matching the visible image and the electronic image to identify the infrared image pixels corresponding to the visible image pixels identified in step S3.

15. The temperature measurement method of claim 13, further comprising application of a predetermined compensation coefficient to the temperature value of each image pixel or to the maximum temperature value determined in step S9.

16. The temperature measurement method of claim 13, wherein the first and second thermal sensors are connected to the first and second reference targets, respectively, by means of a first and a second conductive track, step S5 comprising determination of an instantaneous temperature of the first and second conductive tracks.

17. The temperature measurement method of claim 13, further comprising, prior to step S1, a step S0 of determination of a presence of an individual in the predetermined passage, steps S1 to S9 being implemented only when an individual is present in the predetermined passageway.

18. The temperature measurement system of claim 1, further comprising a flashing light adjacent to the infrared camera and configured to draw a gaze of an individual passing through the predetermined passageway.

19. The temperature measurement method of claim 13, wherein steps S1 and S2 are simultaneous.

20. The temperature measurement method of claim 13, wherein step S8 is only applied to the identified infrared pixels.
